(19) United States
(12) Patent Application Publication
(10) Pub. No.: US 2024/0245894 A1
(43) Pub. Date: Jul. 25, 2024

(54) Thermoplastic Elastomer Composition for Closed System Transfer Device

(71) Applicant: Becton, Dickinson and Company, Franklin Lakes, NJ (US)

(72) Inventors: Wen Yuan, Mountain Lakes, NJ (US); Xianhong Feng, Kearny, NJ (US)

(21) Appl. No.: 18/291,974

(22) PCT Filed: Jul. 29, 2022
(86) PCT No.: PCT/US22/38881

§ 371 Date: Jan. 25, 2024

Related U.S. Application Data
(60) Provisional application No. 63/227,570, filed on Jul. 30, 2021.

(51) Int. Cl. A61M 39/00; C08J 5/22; C08L 53/02

(52) U.S. Cl. CPC A61M 39/00; C08J 5/2218; C08L 53/02; A61M 2039/0027; C08J 2353/02; C08J 2423/08

(57) ABSTRACT

A membrane for a closed system transfer device including a material having 40-50% styrenic block copolymer, 0-10% polypropylene, and 45-60% by weight of mineral oil. The membrane may be utilized in any component of a closed system transfer device or system, such as a syringe adapter, patient connector, vial adapter, IV bag spike, etc. The membrane may also be utilized in scenarios where the cannula of the syringe adapter punctures the membrane and remains in the punctured position for an extended period of time, such as one hour or greater.

### CROSS-REFERENCE TO RELATED APPLICATION

The present application claims priority to U.S. Provisional Application Ser. No. 63/227,570, filed Jul. 30, 2021, the entire disclosure of which is hereby incorporated by reference in its entirety.

### BACKGROUND OF THE INVENTION

### Field of the Disclosure

The present disclosure relates generally to a thermoplastic elastomer composition for a closed system transfer device.

### Description of the Related Art

Health care providers reconstituting, transporting, and administering hazardous drugs, such as cancer treatments, can put health care providers at risk of exposure to these medications and present a hazard in the health care environment. Unintentional chemotherapy exposure can affect the nervous system, impair the reproductive system, and bring an increased risk of developing blood cancers in the future. Some drugs must be dissolved or diluted before they are administered, which involves transferring a solvent from one container to a sealed vial containing the drug in powder or liquid form, by means of a needle. Drugs may be inadvertently released into the atmosphere in gas form or by way of aerosolization, during the withdrawal of the needle from the vial and while the needle is inside the vial, if any pressure differential between the interior of the vial and the surrounding atmosphere exists. In order to reduce the risk of health care providers being exposed to toxic drugs, the transfer of these drugs is accomplished utilizing a closed system transfer device or system.

Closed system transfer devices or systems may utilize membranes to ensure the safe transfer of fluid between components. For example, a syringe adapter may include a membrane that contacts a membrane of a mating component, such as a patient connector, IV bag spike, or vial adapter. The membrane, which may be formed from a thermosetting isoprene rubber, is pierced by a needle of the syringe adapter. Accordingly, the membrane is required to meet scaling and leakage requirements while also limiting membrane fragmentation, which can create small material particles when the needle pierces through the membrane that can pose a risk to a patient. A lubricating agent, such as silicone oil, can be applied to the needle surface and the membrane to minimize membrane fragmentation. The use of a lubricating agent on the surface of the needle and membrane, however, can affect leakage performance, fragmentation, and flow rate through the syringe adapter.

### BRIEF DESCRIPTION OF THE DRAWINGS

The above-mentioned and other features and advantages of this disclosure, and the manner of attaining them, will become more apparent and the disclosure itself will be better understood by reference to the following descriptions of aspects of the disclosure taken in conjunction with the accompanying drawings, wherein:

FIG. 1 is a front view of a patient connector according to one aspect or embodiment of the present application;

FIG. 2 is a cross-sectional view of the patient connector of FIG. 1;

FIG. 3 is a cross-sectional view of the patient connector of FIG. 1, showing the patient connector being inserted into a syringe adapter;

FIG. 4 is a cross-sectional view of the patient connector of FIG. 1, showing the patient connector inserted into a syringe adapter;

FIG. 5 is a chart showing membrane fragmentation final score and mean test results;

FIG. 6 is a chart showing membrane fragmentation individual value plots in category 2 (50 um≤x≤100 um) and category 3 (x>100 um) counted particle numbers and the mean; and

FIG. 7 is a chart showing a scatterplot of fragmentation mean final score versus oil percentage.

Corresponding reference characters indicate corresponding parts throughout the several views. The exemplifications set out herein illustrate exemplary aspects of the disclosure, and such exemplifications are not to be construed as limiting the scope of the disclosure in any manner.

### DETAILED DESCRIPTION

The following description is provided to enable those skilled in the art to make and use the described aspects contemplated for carrying out the invention. Various modifications, equivalents, variations, and alternatives, however, will remain readily apparent to those skilled in the art. Any and all such modifications, variations, equivalents, and alternatives are intended to fall within the spirit and scope of the present invention.

For purposes of the description hereinafter, the terms “upper”, “lower”, “right”. “left”, “vertical”, “horizontal”, “top”, “bottom”, “lateral”, “longitudinal”, and derivatives thereof shall relate to the invention as it is oriented in the drawing figures. However, it is to be understood that the invention may assume various alternative variations, except where expressly specified to the contrary. It is also to be understood that the specific devices illustrated in the attached drawings, and described in the following specification, are simply exemplary aspects of the invention. Hence, specific dimensions and other physical characteristics related to the aspects disclosed herein are not to be considered as limiting.

Unless otherwise indicated, all ranges or ratios disclosed herein are to be understood to encompass the beginning and ending values and any and all subranges or subratios subsumed therein. For example, a stated range or ratio of “1 to 10” should be considered to include any and all subranges or subratios between (and inclusive of) the minimum value of 1 and the maximum value of 10; that is, all subranges or subratios beginning with a minimum value of 1 or more and ending with a maximum value of 10 or less.

The terms “first”, “second”, and the like are not intended to refer to any particular order or chronology, but refer to different conditions, properties, or elements.

As used herein, “at least one of” is synonymous with “one or more of”. For example, the phrase “at least one of A, B, and C” means any one of A, B, or C, or any combination of any two or more of A, B, or C. For example, “at least one of A, B, and C” includes one or more of A alone; or one or more of B alone; or one or more of C alone; or one or more of A and one or more of B; or one or more of A and one or more of C; or one or more of B and one or more of C; or one or more of all of A, B, and C.

In one aspect or embodiment of the present application, a membrane 10 for a closed system transfer device includes a material having 40-50% styrenic block copolymer, 0-10% polypropylene, and 45-60% by weight of mineral oil. The membrane 10 may be utilized in any component of a closed system transfer device or system, such as a syringe adapter, patient connector, vial adapter, IV bag spike, etc. The membrane 10 may be utilized with the syringe adapter shown and described in United States Patent Application Publication No. 2015/0297454, which is hereby incorporated by reference in its entirety.

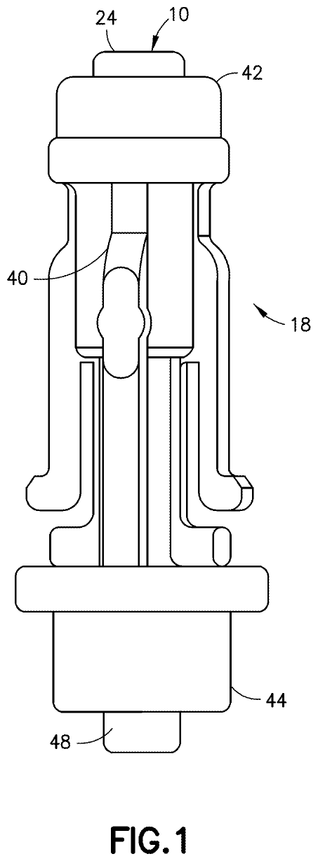

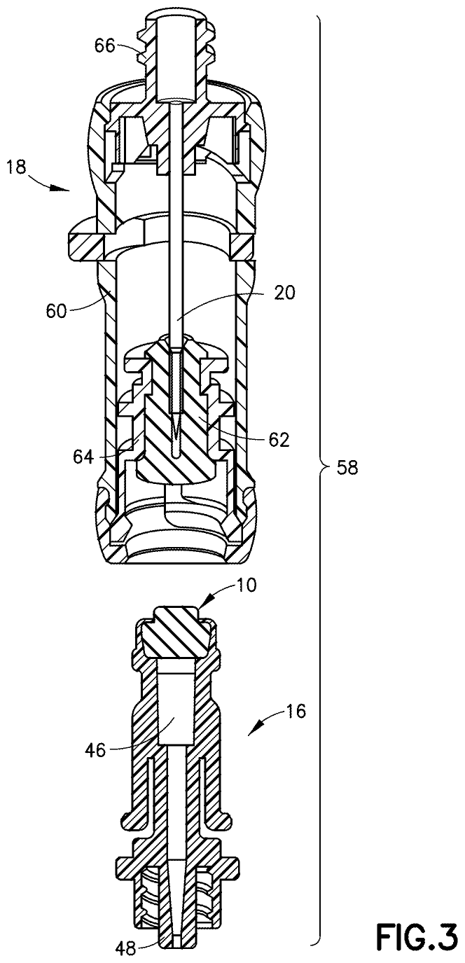

Referring to FIGS. 1-4, the membrane 10 is shown in connection with a patient connector 16, which is utilized to connect one component of a closed system transfer device or system to a patient intravenous line. For example, the patient connector 16 may be connected to a syringe adapter 18 to facilitate the transfer of fluid from one container, such as a syringe barrel, to another container or line, such as an intravenous line, IV bag, or other component. The membrane 10 is configured to prevent leakage through the membrane 10 when the membrane 10 is punctured by a cannula 20. During use, the cannula 20 of the syringe adapter 18 may puncture the membrane 10 and quickly, such as a time period of 10 seconds or shorter, be withdrawn from the membrane. The membrane 10 may also be utilized in scenarios where the cannula 20 of the syringe adapter 18 punctures the membrane 10 and remains in the punctured position for an extended period of time, such as one hour or greater. The membrane 10 is configured to prevent leakage through the membrane 10, such as through an opening caused by the cannula 20 puncturing the membrane 10 or through an interface between the cannula 20 and the membrane 10. A top surface 24 of the membrane 10 is configured to engage a counterpart membrane of another component, as discussed below. The membrane 10 may include a flange 28 as well as other features and structures.

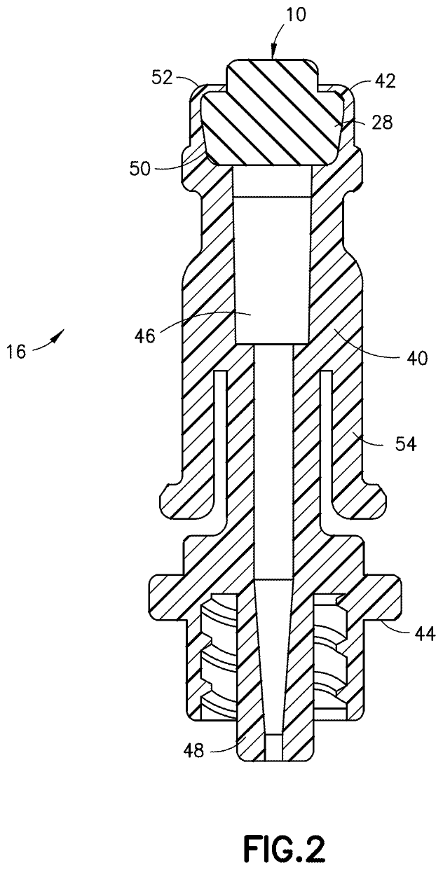

Referring again to FIGS. 1 and 2, the patient connector 16 includes a body 40 having a first end 42 and a second end 44, with the body 40 defining a passageway 46, a line connection 48 positioned at the second end 44 of the body 40, and the membrane 10 positioned at the first end 42 of the body 40. The line connection 48 may be a luer lock connection, although other suitable connections may be utilized. The membrane 10 is received by an opening 50 defined by the body 40 of the patient connector 16. The opening 50 of the patient connector 16 is wider than the passageway 46. The body 40 of the patient connector 16 includes a securing extension 52 at the first end 42 of the body 40, with the securing extension 52 extending radially inward and configured to secure the membrane 10 to the body 40 of the patient connector 16. The patient connector 16 also includes a locking arrangement 54 configured to secure the patient connector 16 to the syringe adapter 18.

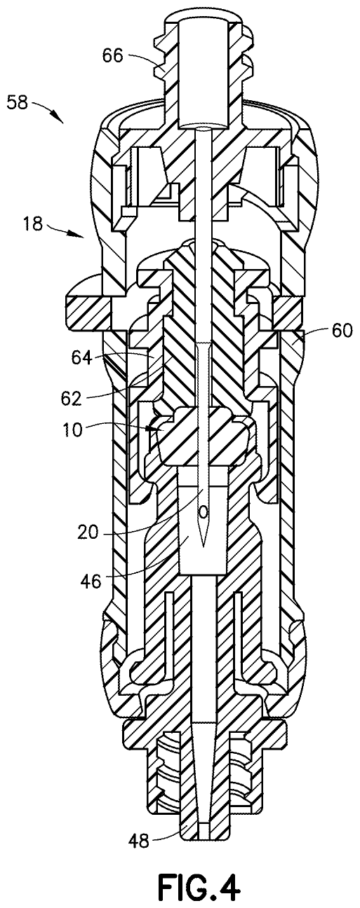

In a further aspect or embodiment, a system 58 for the closed transfer of fluid includes the patient connector 16 and the syringe adapter 18, although the system 58 may also include other components of a closed system transfer device or system. The syringe adapter 18 includes a housing 60 having a syringe adapter membrane 62 received within the housing 60 and the cannula 20. The syringe adapter membrane 62 is moveable from a first position within the housing 60 of the syringe adapter 18 to a second positon within the housing 60 when the patient connector 16 is positioned within the housing 60 of the syringe adapter 18, as shown in FIG. 4. The membrane 10 of the patient connector 16 is configured to engage the syringe adapter membrane 62. The cannula 20 is configured to puncture the membrane 10 of the patient connector 16 and the syringe adapter member 62 when the patient connector 16 is positioned within the housing 60 of the syringe adapter 18. The syringe adapter membrane 62 is received by a collet 64, although other suitable arrangements may be utilized. The syringe adapter 18 includes a luer connector 66 configured to be secured to a syringe barrel. The operation of the syringe adapter 18 is described in United States Patent Application Publication No. 2015/0297454. Accordingly, the membrane 10 and the syringe adapter member 62 needs to maintain a seal to form a closed system while also minimizing fragmentation of the material during puncturing of the membranes 10, 62 with the cannula 20.

One potential solution for a membrane to meet fragmentation and sealing requirements is to apply lubricant or other low surface energy polymer such as fluoropolymer or silicone emulsion directly on the surface of the membrane 10 by spray coating or soaking techniques to lower the friction between needle and membrane. With a limitation on migrating polymer intrinsically, this method will not be applicable for a thicker membrane, and will not meet the multi penetration usage and complex manufacturing step. Another solution is to use thermoplastic elastomer (TPE). TPEs are like synthetic rubber elastomers in that they are elastic; however, they do not rely on a permanent crosslinked structure for the elastic properties. In turn, this allows for TPE properties to be optimized though formulation and compounding while also providing benefits such as better recyclability and manufacturing efficiency. In addition, the benefits of switching to TPE from isoprene rubber include there will be fewer product requirement tradeoffs, as TPE is more easily tunable in composition for desired material properties.

In one aspect or embodiment, the membrane 10 is provided with a high loading of mineral oil with a range of 40-63%. The resulted TPE materials improved intrinsic lubricity from mineral oil while maintaining the other critical mechanical properties including hardness, tensile, tear and compression set, etc., with right selection as well as proper amount addition of styrenic block copolymer (SBC) and polypropylene (PP). Although discussed in connection with the membrane 10, the materials discussed below may be utilized for the syringe adapter member 62 or any other membrane utilized in a closed system transfer device.

The TPE materials were evaluated through both material characterization and product performance evaluation. For example, Table 1 listed six TPE materials' composition with a hardness range from Shore A 25 to 38 for closed system transfer device membrane application evaluations, such as applications in the membrane 10 or the syringe adapter member 62. Their material properties including tensile, tear, compression set and tan delta have been characterized with standard method and summarized in Table 2.

TABLE 1
List of TPE with hardness and oil % results.

| | Hardness | Oil | Polymer % |
|---|---|---|---|
| Materials | Shore A | % | (SBC + PP) |
| TPE-1 | 38 | 40 | 60 |
| TPE-2 | 28 | 43 | 57 |
| TPE-3 | 27 | 50 | 50 |
| TPE-4 | 38 | 58 | 42 |
| TPE-5 | 30 | 60 | 40 |
| TPE-6 | 25 | 63 | 37 |

TABLE 2
Summary of Materials Properties of TPEs

| | | | | | DMA- |
|---|---|---|---|---|---|
| | Tensile | | Compression | Compression | 25 C/1 |
| | at | Tear | set 22 | set 96 hr | Hz |
| | break/ | resistance | hr 23 C | 23 C | Tan |
| Materials | Mpa | kN/m | 1% | 1% | delta |
| TPE-1 | 8.76 | 26.3 | 12.0% | 17.2% | 0.062 |
| TPE-2 | 5.52 | 14 | 8.0% | 19.4% | 0.049 |
| TPE-3 | 4.8 | 15.8 | 7.2% | 10.6% | 0.059 |
| | | | | (72 hr) | |
| TPE-4 | 10.2 | 25.9 | 16.9% | 16.3% | 0.040 |
| TPE-5 | 6 | 15 | 14.0% | 16.6% | 0.068 |
| TPE-6 | 3.8 | 9.8 | 13.0% | 19.3% | 0.079 |

After material characterization, all six TPEs have been molded to the membrane parts and assembled to the final product for performance evaluation including leakage and fragmentation. As shown in Table 3, TPE 3-5 passed all the requirements, while other TPEs outside this composition window cannot meet all the critical requirements for closed system transfer device applications. Given all the product testing results, it is concluded that a softer TPE with ideal composition including mineral oil % from 45% to 60% in weight and a total polymer % including SBC and PP from 40% to 50% and more specifically with an SBC of 40%˜50% and PP of 0˜10% without fillers will fit best for closed system transfer device membrane applications.

TABLE 3
Summary of Product testing results, Hardness and TPE Oil %

| Materials | Leakage | Fragmentation | Oil % |
|---|---|---|---|
| TPE-1 | Fail | Pass | 40 |
| TPE-2 | Fail | Pass | 43 |
| TPE-3 | Pass | Pass | 50 |
| TPE-4 | Pass | Pass | 58 |
| TPE-5 | Pass | Pass | 60 |
| TPE-6 | Fail | Pass | 63 |

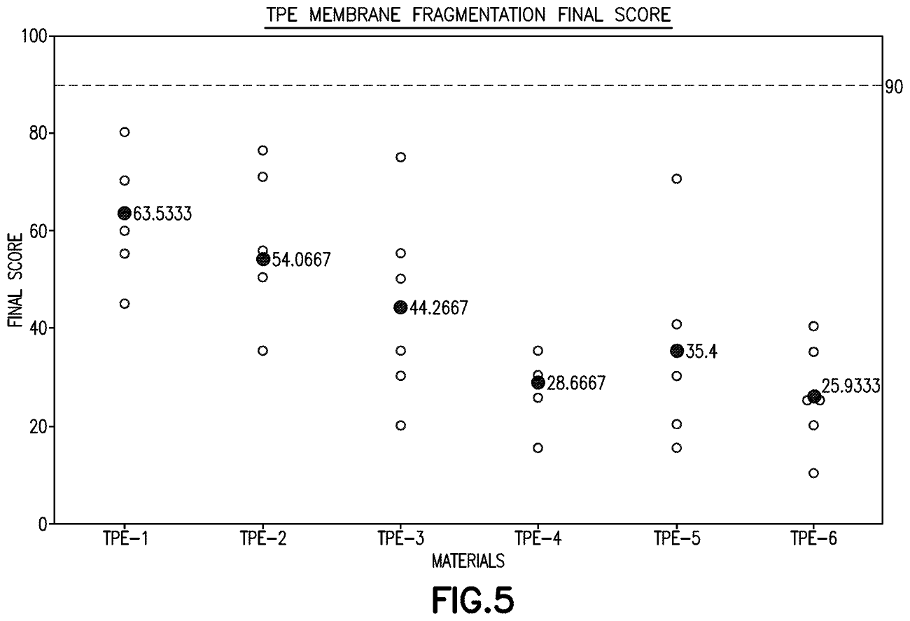

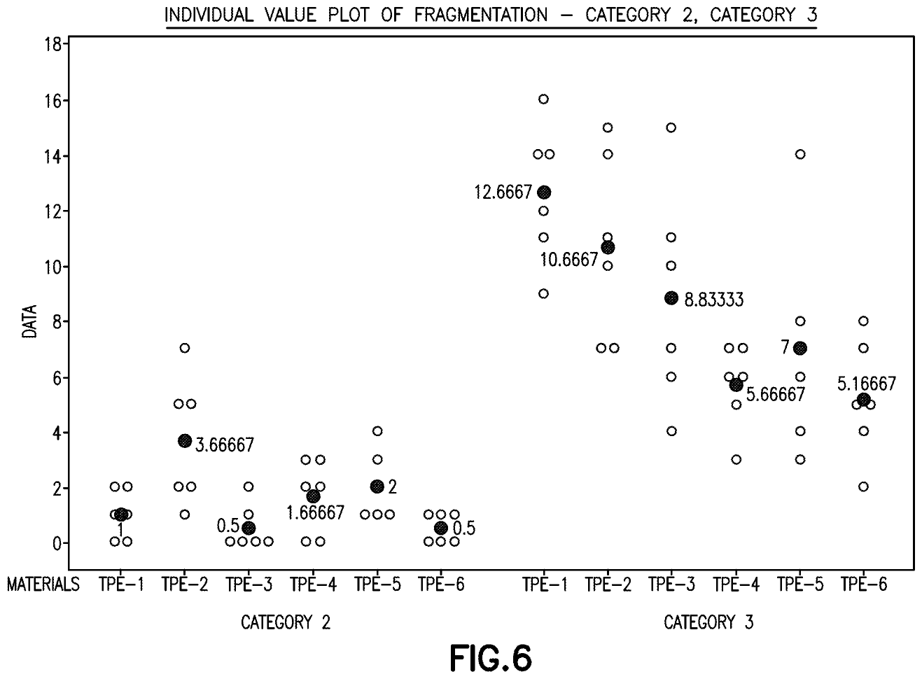

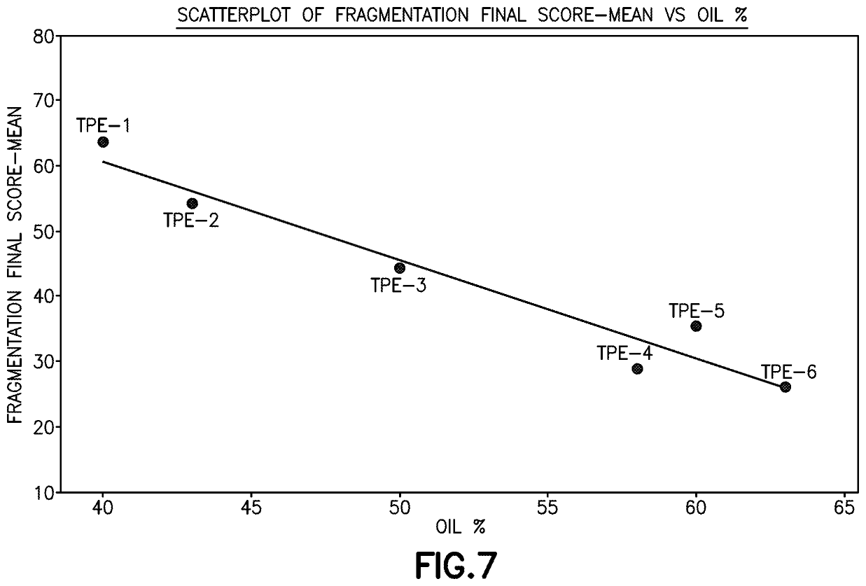

The higher concentrations of oil in the new TPE formulation also showed improved fragmentation performance of the product as shown in FIG. 5 where all six TPEs passed the fragmentation without applying any silicone lube on the needle and membrane pocket. TPE 6 with the highest oil loading of 63% showed the best fragmentation performance among all the candidates. In FIG. 6, it is also shown that TPE 6 performed the best on Category 3 with the least particle number >100 um while TPE 1 with the lowest oil % of 40% generated the most particle numbers in the same category. Further analysis in FIG. 7 exhibited a linear relationship between TPE oil % and fragmentation score in mean, which has also approved a similar relationship between TPE oil % and fragmentation Category 3 particle number in mean (FIG. 6). Thus, the fragmentation performance has been strongly correlated to TPE's oil %.

However as shown in Table 3, too much oil in the TPE formulation such as TPE-6 will result in low mechanical strength including hardness, tensile, tear, compression set, and tan delta, etc., which will compromise TPE membranes' sealing capability. Too little oil in the TPE formulation such as TPE-1 and TPE-2 will result in higher hardness and lower elasticity of TPE material, which also will compromise TPE membranes' sealing capability as well as needle penetration force for closed system transfer device components. The new TPE composition contain a mineral oil % from 45% to 60% in weight and a total polymer % including SBC and PP from 40% to 50% and more specifically with an SBC of 40%-50% and PP of 0-10% without fillers will fit best for closed system transfer device membrane applications.

A newly formulated thermoplastic elastomer as sealing component of closed system transfer device components provide the unique properties benefit for sealing applications, including: 1) unique composition, including 45-60% oil, 40-50% SBC and 0-10% PP, provide a well-balanced mechanical property including hardness (Shore A 32.5±6), tensile (>4 Mpa), tear (>15 kNm), 96 hr compression set (<17%), tan delta (<0.07) to meet all the requirements including leakage and fragmentation for needle penetrable sealing component applications; 2) significantly reduce fragments generation for needle penetrable septum application with higher loading of mineral oil to improve safety and efficacy of closed system transfer device products for a better fragmentation performance; and 3) eliminates silicone oil usage on the needle surface as well as in the membrane pocket to prevent drug interactions with silicone oil and potentially increase the flow rate of drug delivery systems.

While this disclosure has been described as having exemplary designs, the present disclosure can be further modified within the spirit and scope of this disclosure. This application is therefore intended to cover any variations, uses, or adaptations of the disclosure using its general principles. Further, this application is intended to cover such departures from the present disclosure as come within known or customary practice in the art to which this disclosure pertains and which fall within the limits of the appended claims. To the extent possible, one or more features of any aspect or embodiment discussed above can be combined with one or more features of any other aspect or embodiment.

1. A membrane for a closed system transfer device comprising:
a material comprising 40-50% styrenic block copolymer, 0-10% polypropylene, and 45-60% by weight of mineral oil.

2. The membrane of claim 1, wherein the material has a Shore A hardness of 26.5-38.5.

3. The membrane of claim 1, wherein the material has a tensile strength greater than 4 Mpa.

4. The membrane of claim 1, wherein the material has a tear resistance of greater than 15 kNm.

5. The membrane of claim 1, wherein the material has a 96 hour compression set of less than 17%.

6. The membrane of claim 1, wherein the material has a tan delta of less than 0.07.

7. The membrane of claim 1, wherein an outer surface of the material is free from silicone oil.
